SEQUENCE LISTING (4 sequences)

SEQ ID NO: 1 - DNA, 20 bp, Artificial Sequence
  Description of Artificial Sequence antisense rat specific
tgacgcctccggagctgaga

SEQ ID NO: 2 - DNA, 20 bp, Artificial Sequence
  Description of Artificial Sequence antisense human specific
tgacgcctctggagctgagc

SEQ ID NO: 3 - DNA, 30 bp, Artificial Sequence
  Description of Artificial Sequence  primer for PCR amplification product
cgcggatccatggctgagtacgggaccctc

SEQ ID NO: 4 - DNA, 43 bp, Artificial Sequence
  Description of Artificial Sequence primer for PCR amplification product
ggccttcttcggtgggggagccaatttgatgatctcttcctca

(19) United States
(12) Patent Application Publication
(10) Pub. No.: US 2007/0028311 A1
(43) Pub. Date: Feb. 1, 2007

(54) Therapeutic and diagnostic means for papillomas and other diseases involving ped/pea-15

(76) Inventors: Francesco Beguinot, Napoli (IT); Claudia Miele, Napoli (IT); Pietro Formisano, Portiel (IT)

Correspondence Address: NIXON & VANDERHYE, PC, 901 NORTH GLEBE ROAD, 11TH FLOOR, ARLINGTON, VA, 22203, US

(21) Appl. No.: 10/559,627

(22) PCT Filed: Jun. 3, 2004
(86) PCT No.: PCT/IT04/00325

§ 371 Date: Dec. 6, 2005

(30) Foreign Application Priority Data
Jun. 6, 2003 (IT) RM2003A000283

(51) Int. Cl. A01K 67/027; C12Q 1/68; C12N 5/06

(52) U.S. Cl. 800/10; 435/6; 800/18; 435/354

(57) ABSTRACT

The present invention discloses diagnostic methods and means, as well as medicaments in diagnosis, prognosis and therapy of diseases where PED/PEA-15 plays an etiopathogenetic role, in particular papilloma and diabetes. Said methods are based on the determination of PED/PEA-15 levels and the medicaments are based on antisense oligonucleotides. A non-human transgenic animal, expressing high PED/PEA-15 levels, is also disclosed, being it useful as a model for the study of said diseases and evaluation of their therapies.

The present invention relates to means for the treatment and diagnosis of papilloma and other diseases involving PED/PEA-15, in particular to compounds useful as medicaments for their therapy and to transgenic animals useful for their study and diagnosis.

### BACKGROUND OF THE INVENTION

Neoplastic cells, through the transformation, acquire both excessive proliferation and abandonment of their ability to die (Duke, R., et al. (1996): Sci. Am. 275: 80-87). Cancer develops after a cell accumulates mutations in several genes controlling proliferation and survival (Duke, R., et al. ibid; Thompson, C. B., (1995): Science 267: 1456-1472). When a mutation cannot be repaired, the affected cell usually runs death programs (apoptosis). However, if the cell does not die, said cell (or its progeny) may live long enough to accumulate mutations that make it possible to divide uncontrollably and to metastasize (Duke, R., et al. ibid). In many solid tumors, including lung, colon and breast, the genetic damage affects p53, a protein required for cells to initiate apoptosis when DNA is injured (Thompson, C. B., ibid). Other apoptosis-related proteins have also been implicated in human malignancies. Thus, in certain lymphomas, cell death is blocked by excessive production of the anti-apoptotic factor BCL-2 (Thompson, C. B., (1995): ibid). And there are evidences that some tumors prevent Fas from conveying signals to the death machinery, or produce Fas ligand to avoid immuine-mediated apoptosis (Duke, R., et al. ibid).

Cells that become cancerous might also be dysregulated through other triggers. The tendency of normal cells to commit suicide when they are deprived of their growth factors or of cognitive interactions with their neighbours has probably been established as a built-in defence against metastasis (Duke, R., et al. ibid); prompt activation of apoptosis in tumor cells that leave their native tissue presumably eradicates many metastatic cells before they have a chance to grow. Unfortunately, cancer cells often become resistant to the apoptotic effect of growth factor deprivation and loss of cell-to-cell contact.

This last event makes an important problem in the treatment of cancer, where one goal is to restore naturale apoptosis mechanism of a cell, whose genetic code was damaged, causing uncontrolled proliferation.

Altered apoptotic programs in cells also have an important role in acquiring resistance to radiation and chemotherapy effect. In fact, many, of these agents exert their action by inducing damages in DNA and activating p53, which, having recognized damage, triggers apoptosis (Duke, R., et al. ibid). Therefore, cells that lacks p53 or that produce high levels of the BCL-2 inhibitory protein can develop resistance to the effects of a number of anti cancer drugs.

Also this phenomenon comes true in a practical problem, since many antitumor therapies fail right because of the development of resistance by cancer cells.

Receptors for cytokines of the tumor necrosis factor (TNF) family can also induce apoptosis (Green D. R., (1998): Cell 94: 695-698). The best characterized among these receptors and their cognate ligands include TNFR1/TNF-α, 0Fas/FasL, TRAIL-R/TRAIL and DR3/TWEAK (Ashkenazi, A. et al. (1999): J. Clin. Invest. 104: 155-162). Targeting these death receptors provides a promising therapeutic strategy for cancers (Walczak, H., et al., (1999): Nat. Med. 5: 157-163; Ashkenazi, A., et al. (1999): J. Clin. Invest. 104: 155-162). For instance, TRAIL administration has been shown to suppress growth of a number of tumor types in animals with little toxicity (Walczak, H.; Ashkenazi, A., ibid).

The present inventors have recently addressed this issue by examining a large panel of human malignant glioma cell lines for their sensitivity to TRAIL. The present inventors have shown that TRAIL, but not, TNF-α, FasL, or TWEAK, can induce apoptosis in malignant glioma cells (Hao, C., et al., (2000): Brain Path. 4:730; Hao, C., et al., (2001): Cancer Res. 61: 1162-1170). TRAIL is a recently identified cytokine that triggers rapid apoptosis in various tumor cells (Wiley, S., et al. (1995): Immunity, 3: 673-683). TRAIL can interact with two death receptors, TRAIL-R1 and TRAIL-R2, and two decoy receptors, TRAIL-R3 and TRAIL-R4 (Pan, G., et al., (1997): Science 276: 111-113, Pan, G., et al., (1997): Science 277: 815-818; Walczak, H., et al., (1997): EMBO J 16: 5386-5397; Chaudhary, P. M., et al., (1997). Immunity 7: 821-830; Sheridan, J. P., et al., (1997): Science 277: 818-821; Pan, G., et al,. (1998): FEBS Lett. 424: 41-45, Ma-rsters, S. A., et al., (1997): Curr. Biol. 7: 1003-1006). TRAIL-R1 and TRAIL-R2 contain an intracellular motiftermed the death domain (DD) that is necessary for activating caspase-8 and the downstream caspase cascade leading to apoptosis (Pan, G., et al., (1997): Science 276: 111-113). Similar to Fas and TNFR1, activation of the caspase cascade by TRAIL-R1 and 2 involves receptor engagement by the TRADD and FADD adapter proteins (Walczak, H., et al., (1997): EMBO J. 16: 5386-5397; Chaudhary, P. M., et al., (1997): Immunity 7: 821-830). FADD, in turn, engages caspase-8 through its death effector domain (DED), activating the caspase cascade (Cryns, V. and Yuan, J., (1998): Genes and Development 12:1551-1570). There is also evidence, however, that FADD independent pathways are involved in TRAIL apoptosis (Sheridan, J. P., et al., (1997): Science 277: 818-821). Moreover, TRAIL-induced apoptosis in human cells involves JNK function (Herr, L, et al., (1999): Cell Death and Differentiation 6: 130-135). In contrast to TRAILR-1 and TRAIL-R2, TRAIL-R3 and TRAIL-R4 are unable to transduce the death signal (Pan, G., et al., (1997): Science 277: 815-818; Sheridan, J. P., et al., (1997): Science 277: 818-821; Marsters, S. A., et al., (1997). Curr. Biol. 7: 1003-1006), suggesting that decoy receptors may perform anti-apoptotic functions. Recent studies, however, have shown that TRAIL-R3 and TRAIL-R4 expression is relatively minor in many tumor cells and TRAIL-induced apoptosis is firstly regulated intracellularly (Griffith, T. S., et al., (1998): J. Immunol. 161: 2833-2840; Leverkus, M., et al., (2000): Cancer Res. 60: 553-559). It is not yet clearly understood how this regulation occurs.

Therefore, TRAIL can be thought as an important therapeutic mean for tumor treatment.

The present inventors have recently cloned a death effector domain (DED)-containing intracellular protein, which is prominently expressed in astrocytes (Condorelli, G., et al., (1998): EMBO J. 17: 3858-66). We named this 15 KDa novel protein PED/PEA-15. First cloning studies on PED/PEA-15 revealed the presence of the DED and of three PKC consensus phosphorylation sites (only one is phosphorylated by PKC in intact cell (Herr, I., et al., (1999): Cell Death and Differentiation 6: 130-135; Griffith, T. S., et al., (1998): J. Immunol. 161: 2833-2840). However, these studies provided no clue on the potential function of its protein transcript. We found however that, in different cell types, PED blocks FAS and TNFR1-triggered apoptosis by inhibiting FADD-caspase-8 binding through its DED (Condorelli, et al., (1999): Oncogene 18: 4409-4415). PED blocks also TRAIL-induced apoptosis in human glioma cells (Hao C., et al., (2001). Cancer Res 61: 1162-1170). The mechanism by which PED exerts this action was deeply investigated by the present inventors (Xiao, C., et al., (2002): J. Biol. Chem. 277: 250 20-25025). At variance with other known proteins which inhibit FADD activation of caspase-8, PED also prevents apoptosis following growth factor starvation and exposure to UV light and osmotic stimuli, indicating that PED performs a broad anti-apoptotic function in the cell (Condorelli, G., et al., (2001): Diabetes 50: A298). In fact, our more recent investigations showed PED/PEA-15 is able to block very early apopiotic signals transmitting through JNK and p38 pathways (Condorelli, G., et al. (2001): J. Biol. Chem. 277: 11013-11018).

In cooperation with Dr. C. Hao (Alberta University, Edmonton, Canada), the present inventors have also found that PED/PEA-15 is over-expressed by half human glioma cell lines, thus suggesting a role for PED in gliomagenesis (Hao, C., et al., 2000 and 2001, see above). In glioma cell lines, the levels of PED expression closely correlate with resistance to TRAIL-induced apoptosis, so that cells having low PED/PEA-15 levels are TRAIL sensitive, whereas cells having high levels of PED/PEA-15 levels are TRAIL resistant (Hao, C., et al., 2000 and 2001, vedi so-pra). In addition, we could also demonstrate that transfecting PED/PEA-15 cDNA in TRAIL-sensitive cells makes them resistant; on the contrary, expressing a PED/PEA-15 antisense in TRAIL-resistant cells reduces PED level in cells and induces simultaneously the recovery of sensitivity to TRAIL (Hao, C., et al., 2000, vedi sopra). PED-induced TRAIL resistance can give a contribution to tumorigenesis in gliomas and other tumors.

Moreover, the determination of PED level in human gliomas could allow identification of those gliomas sensitive to TRAIL therapy, an important goal as far as present therapy chances have not significantly improved survival of malignant glioma bearing patients.

PED is overexpressed in mouse metastatic squamous carcinoma cells (Dong, G., et al., (2001): Cancer Res. 61: 4797-4808).

It has now surprisingly been found that PED is also well expressed in human keratinocytes.

In order to investigate whether PED overexpression plays a role in multistage carcinogenesis, the present inventors obtained a transgenic animal, a mouse, overexpressing PED/PEA in the skin. Such an animal is useful for studies on cutaneous tumorigenesis (topical treatment with DMBA, followed by multiple TPA exposures).

It was suprisingly observed that transgenic mice develop a number of papillomas significantly higher than control animals.

Further, PED/PEA-15 expression significantly increases in papillomas with respect to normal skin, both in transgenic animals and in control ones, suggesting an important role for PED/PEA-15 in determining susceptivity in cutaneous carcinogenesis. Lesions developing in transgenic animals develop, with a significantly higher frequency, toward carcinoma stage. Finally, carcinomas developed in transgenic animals are histologically more aggressive.

These observations cannot be inferred from state of the art knowledge, nor from those about cutaneous papilloma, or about PED/PEA-15.

The person skilled in this art is aware that PED/PEA-15 expression levels in human tissues are not homogeneous. In addition to this fact, it is really doubtful whether tumors express higher protein levels with respect to normal tissue, therefore, the person skilled in this art cannot foresee that papillomas express PED levels. In fact, to the present knowledge, PED cannot be considered a general marker for tumors. Finally, apoptosis, which is the most pertinent event to PED/PEA-15 natural activity, does not concern cutaneous tumorigenesis, maybe is even not related to tumorigenesis.

Relying on whole complex of present knowledge about PED/PEA-15, it will be evident to the skilled person that there is neither logical relation available nor a clear indication, with a reasonable expectation of success that it would be possible to find PED/PEA-15 expressed in papillomas nor the therapeutic and diagnostic possibilities that the discovery at the basis of the present invention provides.

Other than the above mentioned references, also the paper by Tsukamoto, T., et al., Cancer Lett. 2000 Feb. 28; 149 (1-2): 105-13 is to be cited, which discloses MAT1, a transformant gene, which was cloned from a mouse mammary tumor and which was seen to be identical to the untranslated 3′ region of the 2.5 kb PEA-15 isoform. An aberrant expression of MAT1/PEA-15 isoforms was found in mouse breast epithelial cell lines. It is well-known that epithelia are really different from papillomas.

To inventors' knowledge, the only industrial application of the information available on PED/PEA-15 is provided by international application WO 02/22867, to Evotec Neurosciences GMBH, published on 21 Mar. 2002, which discloses a method for the diagnosis or prognosis of Alzheimer's disease or other neurodegeneratives diseases based on the determination of the PEA-15 level and the treatment of said diseases with a protein inhibitor.

The state of the art makes evident that PED/PEA-25 is not univocally associated to tumors. In fact, former studies by the present inventors established a role of this protein in type 2 diabetes, a pathology totally unrelated with tumors. (Condorelli, et al., EMBO J., 1998, July 15, 17(14): 3858-66)

Data obtained by the present inventors show that PED is well expressed in human keratinocytes. Moreover, another reference (Dong, G., et al., (2001): Cancer Res. 61: 4797-4808), directed to random identification of differentially expressed genes in mouse squamous carcinoma cell lines, shows PED overexpression, together with differences in expression of other 72 genes. The state of the art does not allow to foresee in any way the functional meaning of PED overexpression in cutaneous tumorigenesis. To completeness of the above mentioned study, it is to be firstly underlined that there is no available functional analysis related to disclosed altered genic expression profile and, secondly, the mentioned paper considers cell lines, which is a well-known different matter from cells of a histologically defined neoplasia. However, the state of the art allows to identify minimal requirements for verifying the possibility that PED has a role in the risk of neoplastic transformation of cutaneous elements, such as, for example, its expression in some normal cutaneous cell elements. It must also be said that cutaneous tumorigenesis, induced through carcinogenic chemical compounds is a widely used model for the study of molecular mechanisms of transformation. This implies that the role of a candidate gene, ped in this case, in the cutaneous tumorigenesis mechanism can be defined with higher accuracy in the cutaneous model with respect to other systems.

As above mentioned, transgenic animal, a mouse, obtained by the present inventors, shows a susceptibility increase to cutaneous tumor risk, and this is a surprising finding for at least two reasons. The first one is that the differences of risk of tumor between transgenic and wild-type mice (and the contour data, such as coherent correlations between PED expression levels and tumor regression or tumor malignancy degree) are sufficiently ample to achieve statistical significance even with a low number of animals. The second reason is that the cutaneous tumorigenesis subsequent to the application of chemical carcinogens is an accepted example of multistage carcinogenesis, and, as such, a phenomenon linked to the participation at the same time of discrete number of events. This fact cannot allow to infer that a single event (a single gene overexpression, ped in this case) can so heavily affect the whole evolution of neoplastic process, from papilloma to carcinoma in its different malignancy degrees.

Finally, the above mentioned ped antiapoptotic function does not allow the skilled person to conclude that cutaneous tumorigenesis is a direct consequence of its role. Indeed, apoptosis alterations have a well-defined role in some, but not all tumors. As above described, cutaneous tumors do not represent, at the present state of the art, a reliable example of transformation wherein apoptosis defects have an established role in their pathogenesis. More generally, the concept that the neoplastic transformation is a “multistage” phenomenon must be repeated. Multiple alterations are necessary because the transformed phenotype can be acquired. Therefore, it is not possible to establish αpriori whether and to what extent a specific event, in the specific case ped overexpression can determine the risk of neoplasia.

### ABSTRACT OF THE INVENTION

It is an object of the present invention a method for the diagnosis or prognosis of a papilloma in a subject, or for the determination of the risk for said subject to develop a papilloma, or a method for the monitoring of the progression of a papilloma in a subject, or a method for the evaluation of a therapeutic treatment of a papilloma in a subject.

It is another object of the present invention a non-human recombinant animal comprising a non-native genetic sequence coding for PED/PEA-15, or a fragment or a derivative thereof Preferably, said animal is a mammal, more preferably a mouse.

Another object of the present invention are antisense oligonucleotides of PED/PEA-15 and their use as medicaments. In particular, said antisense oligonucleotides are useful for the preparation of medicaments for the prevention and/or treatment of tumors.

With the term “treatment” it is intended both early treatment of neoplasias, even benign ones, and treatment of neoplasias at an advanced stage.

In one of its preferred embodiments, the present invention refers to papilloma, also of viral origin.

These and other objects of the present invention will be illustrated in higher detail in the following description also by means of examples.

### DETAILED DISCLOSURE OF THE INVENTION

The present invention provides not only methods for diagnosis, prognosis and monitoring of treatments of papilloma, but also a model for the study of papilloma, development of new drugs for its treatment, said model showing reliable and reproducible.

The present invention also provides substances for the preparation of medicaments for the treatment of papilloma overcoming apoptosis resistance phenomenon, developed by some tumors.

In a first embodiment of the invention, the method for the diagnosis or prognosis of papilloma or for the determination of the risk to develop a papilloma, comprises the determination of the level and/or the activity of:
(a) a transcription product of a gene coding for PED/PEA-15, and/or
(b) a translation product of a gene coding for PED/PEA-15, and/or
(c) a fragment or derivative of said transcription or translation product,
in a sample coming from a subject, to whom a papilloma is to be diagnosed or prognosticated, comparing said level and/or activity to a reference value representative of a papilloma or health status (absence or substantial lack of probability of development of a papilloma), then formulating a diagnosis or a prognosis of papilloma in said subject or determining if said subject is at risk of developing a papilloma.

The above-disclosed method can be also applied to monitoring the progression of a papilloma or for evaluating a therapeutic treatment in an affected subject.

According to an embodiment of the invention, the sample to analyse is taken from skin.

In the method according to the present invention, the reference value is the value of the level and/or activity of a transcription product and/or a translation product and/or a fragment or derivative of said transcription or translation product of a gene coding for PED/PEA-15, taken form a sample coming from a subject who is not affected by a papilloma.

The transcription product and/or a translation product and/or fragment or derivative is mRNA and/or the PED/PEA-15 protein and/or a fragment or derivative thereof, respectively.

The determination of the transcription product and/or fragment or derivative is made by means of methods well-known to the person skilled in this art and a further explanation is not necessary. Reference can be made to the above mentioned literature. Preferred examples of said methods are PCR or Northern blot analysis.

In a similar manner, said translation product and/or fragment or derivative can be determined, for example, by means of an immune assay, an enzymatic activity assay and/or a binding assay.

In another embodiment of the invention, the method further comprises the comparison with the above mentioned level and/or activity in a set of samples coming from said subject collected in a period of time. Preferably, the subject receives a therapeutic treatment before the collection of one of the periods of time. Opportunely, the level and/or activity is determined before and after the subject treatment.

The above disclosed methods can be conveniently carried out by means of an assay kit comprising a transcription product and/or a translation product and/or a fragment or derivative of said transcription or translation product of a gene coding for PED/PEA-15. The kit will be prepared with conventional methods, which are well-known in the art and will contain reactants and auxiliary substances useful in the carrying out of the above disclosed methods, for example PCR or Northern blot analysis, an immune assay, an enzymatic activity assay and/or a binding assay.

It is another object of the present invention a non-human recombinant animal, preferably a mammal, in particular a mouse, comprising a non native genetic sequence coding for PED/PEA-15, or a fragment or a derivative thereof. Said animal is obtainable by means of a method comprising:
(a) providing a gene targeting construct comprising said gene sequence and a selectable marker sequence, and
(b) introducing said construct in a stem cell of a non-human animal, and
(c) introducing said stem cell in a non-human embryo, and
(d) transplanting said embryo in a non-human pseudopregnant animal, and
(e) allowing said embryo to develop to term, and
(f) identifying a genetically altered non-human animal whose genome comprises a modification of said gene sequence in both alleles, and
(g) breeding said genetically altered animal to obtain a non-human animal whose genome comprises a modification of said endogenous gene. In a particular embodiment of the invention, a disruption of said gene results in a predisposition to developing a papilloma.

The transgenic animal according to the present invention can express PED/PEA-15 ubiquitously.

By means of techniques available to the person skilled in the art, and using suitable tissue-specific promoters, it is also possible to obtain a transgenic animal expressing PED/PEA-15 specifically or preferentially in a particular tissue. Said animal is a further object of the present invention.

The present invention comprises also the progeny of the transgenic animal and different transgenic lines of the animals obtained by mating with different mice strains, said different transgenic lines being capable of expressing PED/PEA-15.

The transgenic animal according to the present invention is a valid model for studying and verifying pathologies where PED/PEA-15 plays a pathogenetic role. Examples of said pathologies are tumors, for example gliomas, papillomas, also of viral origin, breast cancer, and diabetes, in particular diabetes mellitus, diabetes complications, micro-and macrovascular complications. When suitably treated, the animal of the present invention will have a high probability of developing the pathology, which can also be in a particularly aggressive form. Said non-human animal is an extremely useful model for the study and development of new drugs for the treatment of said pathologies, as well as for the evaluation of efficacy drugs currently used in therapy. The use of this animal in the above mentioned meaning is within the boundaries of the present invention. A particularly preferred embodiment of the invention relates papilloma.

Another object of the present invention is an assay for the screening of a substance useful for the treatment of papilloma comprising:
(a) contacting a biological model of papilloma with said substance;
(b) measuring the activity and/or the level of a second substance selected in the group consisting in a transcription product of a gene coding for PED/PEA-15, and/or a translation product of a gene coding for PED/PEA-15, and/or a fragment or derivative of said transcription or translation product,
(c) measuring the activity and/or the level of said second substance in a control biological sample, which was not contacted with said substance;
(d) comparing the activities and/or levels of steps (b) and (c) and determine whether said substance is an inhibitor of said second substance.

In this context, biological model means any model validly accepted in the field, in particular the transgenic animal of the present invention or any part thereof, for example an organ, tissue or cells affected by the pathology.

The present invention comprises also substances obtainable from the above mentioned assay and their use as active ingredients in the preparation of a medicament for the treatment of papilloma.

In another aspect, the present invention provides antisense oligonucleotides useful as active ingredients in medicaments, in particular for the preparation of a medicament for the, treatment of a pathology where PED/PEA-15 plays a pathogenetic role, for example papilloma or diabetes.

The antisense oligonucleotide according to the present invention is targeted to nucleobase 1 to nucleobase 100 of a nucleic acid molecule encoding PED/PEA-15. Preferably, the antisense oligonucleotide is targeted to sequences encompassing nucleobase 70, 71 or 72 of said nucleic acid molecule encoding PED/PEA-15. Typically, the antisense oligonucleotide is 8 to 30 nucleobases in length. In a preferred embodiment of the present invention, the antisense oligonucleotide is selected from the group consisting of 5′-tgacgcctccggagctgaga-3′ and 5′-tgacgcctctggagctgagc-3′.

Antisense oligonucleotides according to the invention are prepared in a well-known manner. For example, the modified internucleoside linkage is a phosphorothioate linkage and/or the antisense oligonucleotide comprises at least one modified sugar moiety and/or antisense oligonucleotide comprises at least one modified nucleobase. Preferably, the modified sugar moiety is a 2′-o-methoxyethyl sugar moiety or the modified nucleobase is a 5-methylcytosine.

Preferred antisense oligonucleotides according to the present invention are 5′-tgacgcctccggagctgaga-3′ PTO HPS (rat specific) (SEQ ID No. 1); 5′-tgacgcctctggagctgagc-3′ PTO HPS (human specific).

Said oligonucleotides, and the substances obtainable from the above disclosed assay, will be formulated as medicaments in the form of pharmaceutical compositions, which are comprised within the scopes of the present invention.

According to the present invention, the pharmaceutical compositions comprise at least an active ingredient, in an amount sufficient to produce a significant therapeutic effect. The compositions comprised in the present invention are quite conventional and are obtained with methods commonly used in pharmaceutical industry, such as for example shown in Remington's Pharmaceutical Science Handbook, Mack Pub. N.Y.—last edition. According to the selected administration route, the compositions will be in the solid or liquid form, suitable for the oral, parenteral, intravenous, in particular topical route. The compositions according to the present invention comprise together the active ingredient at least a pharmaceutically acceptable vehicle or excipient. Formulation coadjuvant, for example solubilizing, dispersing, suspending, emulsifying agents can be particularly useful.

In another embodiment of the present invention, the pharmaceutical compositions contain a further active ingredient, an antitumor or an agent for the treatment of diabetes, complications thereof, micro- and macrovascular complications. In a preferred embodiment of the invention, said antitumor agent is TRAIL.

The following examples further illustrate the invention.

### EXAMPLE 1

Generation of Ped Transgenic Mice

PED cDNA was cloned in the BamHI sites of the pBap2 plasmid containing the human beta-actin promoter. To generate transgenic mice, the 5.2-kb ClaI fragment was excised, purified by agarose gel electrophoresis and injected into pronuclei of C57BL/6×DBA2 mouse embryos. Three F0 founders were identified by Southern-blot analysis of genomic DNA probing with the PstI fragment of the human beta actin promoter. Heterozygous transgenic mice were identified by: i) Southern-blotting with either the ClaI fragment or the fragment PCR amplification product obtained with the following primers 5′-CGCGGATCCATGGCTGAGTACGGGACCCTC-3′ (SEQ ID No. 3) and 5′-GGCCTTCTTCGGTGGGGGAGCCAATTTGATGATCTCTTCCTCA-3′ (SEQ ID No. 4); ii) Northern-blotting with the ClaI fragment;. iii)

Western-blotting with polyclonal rabbit antibodies toward PED protein.

### EXAMPLE 2

Chemical Cancerogenesis

Mice were subjected to two applications of 100 nmol of 9,10-dimethyl-1,2-benzanthracene (DMBA) in 0.2 ml acetone on their dorsal skin. Upon two weeks, further applications of 5 nmol of phorbol mirystate acetate (PMA) were performed (twice per week for 16 weeks) at the same sites. Upon terminating the treatment, mice were followed up for 16 further weeks and papillomas quantitated weekly, followed by hystopathological analysis (Tables 1-3).

TABLE 1
Chemical cancerogenesis upon DMBA/PMA
treatment in PED+ mice.

| | Number | | # of papillomas/ | # of papillomas/ |
|---|---|---|---|---|
| | of mice | M/F | mouse (8 weeks) | mouse (16 weeks) |
| Tg-PED+ | 18 | 10/8 | 7* | 19* |
| WT | 20 | 10/10 | 3 | 7 |

Tg-PED+: PED transgenic mice; WT: wild-type control mice.
*Differences with WT mice, determined by t-test analysis, are statistically significant (p < 0.01).

TABLE 2
Regression of skin lesions upon termination
of PMA treatment in PED+ mice.

| | # of papillomas/ | # of papillomas/ | # of papillomas/ |
|---|---|---|---|
| | mouse after | mouse after | mouse after |
| | 2 weeks | 8 weeks | 15 weeks |
| Tg-PED+ | 14* | 9* | 6* |
| WT | 4 | 2 | 1 |

Tg-PED+: PED transgenic mice; WT: wild-type control mice.
*Differences with WT mice, determined by t-test analysis are statistically significant (p < 0.01).

TABLE 3
Malignant conversion of papillomatous lesions upon
termination of PMA treatment in PED+ mice.

| | # of carcinomas/lesions | Rate of malignant conversion |
|---|---|---|
| Tg-PED+ | 12/48* | 25% |
| WT | 1/20 | 5% |

Tg-PED+: PED transgenic mice; WT: wild-type control mice.
*Differences with WT mice, determined by t-test analysis are statistically significant (p < 0.01).

### EXAMPLE 3

Comipetitive ELISA assay

Coating—Microwell coating was achieved by recombinant PED incubation for 16h at 4C (100 μl in PBS, 0.5-2.5 μg/ml). Wells were washed three times with 200 μl of PBS supplemented with 0.5% Tween 20 (PBS-T). Saturation of non-specific sites was achieved by a further incubation with 200 μl/well of 3% skimmed milk containing PBS for 2h at 37C followed by 3 additional washings with PBS-T. Quantitation of coating GST-PED is shown in Table 4.

Solid-phase antigen-antibody reaction—For quantitating PED-antibody interaction, the antiserum was diluted as indicated in Table 5 in PBS supplemented with 1% skimmed milk. Diluted antisera were added to the wells (100 ml/well) and incubated for 16h at 4C followed by extensive rinsing with PBS-T.

Colorimetric reaction—Peroxidase-conjugated anti rabbit IgG goat antibodies were added to the wells (1:1500 dilution in PBS containing 3% skimmed milk) and further incubated for lh at 37C. The calorimetric reaction was started by adding 100, μl of a solution containing ophenylenediamine (1mg/ml) and 1μl/ml of 30% hydrogen peroxide in 0.1 M citrate phosphate buffer, pH 5.0. Upon 20 min incubation at 25C, the reaction was blocked by addition of 40 μl/well of sulphuric acid. Optical densities were read at 490 nm.

Liquid-phase antigen antibody interaction—GST-PED standards in 1% skimmed milk PBS were incubated with PED antiserum (1:6400 dilution in 1% milk PBS) for 2h at 37C. Solid phase incubation was subsequently achieved as described above.

TABLE 4
Evaluation of the adequate PED concentration
for microwell coating.

PED concentration
(μg/ml)

| | 0 | 0.5 | 1.25 | 2.50 |
|---|---|---|---|---|
| O.D. 490 nm * | 0.22 | 0.80 | 1.50 | 2.30 |

Ab dilution 1:6400.
* Differences among values were analyzed by t-test and were statistically significant (p < 0.01).

TABLE 5
Evaluation of anti-PED Ab dilution for the immunoenzymatic
detection of PED protein (2.5 μg/ml for well coating).

| | | | | | |
|---|---|---|---|---|---|
| Ab dilution | 1:200 | 1:400 | 1:800 | 1:1600 | 1:3200 |
| O.D. 490 nm | 3.50§ | 3.40§ | 3.45§ | 3.20§ | 2.7*0 |
| Ab dilution | 1:6400 | 1:12800 | 1:25600 | NO Ab | |
| O.D. 490 nm | 2.30* | 0.9* | 0.7* | 0.25* | |

*Differences among values were analyzed by t-test and were statistically significant (p < 0.01).
§Differences were not statistically significant).

TABLE 6
Determination of the standard values for
the quantitative detection of PED.

PED concentration in
liquid phase preincubation
(μg/ml)

| | 0 | 0.5 | 1.25 | 2.50 |
|---|---|---|---|---|
| O.D. 490 nm | 2.30* | 1.85* | 1.25* | 0.65* |

Anti-PED Ab were used at 1:6400 dilution in liquid phase.
*Differences among values were analyzed by t-test and were statistically significant (p < 0.01).

### EXAMPLE 4

Inhibition of PED Expression

Block of PED expression in rat insulinoma (RIN-1) and human glioma (U373MG) cells was achieved by transfection of the following phosphorothioate antisense oligonucleotide (4 μg/60 mm cell dish). 5′-tgacgcctccggagctgaga-3′ PTO HPS (rat-specific); 5′-tgacgcctctggagctgagc-3′ PTO HPS (human-specific). Antisense transfection was accomplished with the Lipofectamine reagent (Invitrogen Cat No. 18324-012) according to the manufacturer's instructions. Quantitation of antisense action on PED protein expression in RIN-1 cells is shown in Table 7.

TABLE 7
Determination of endogenous PED levels in rat insulinoma
cells (RIN-1)and in human glioma cells (U373MG) upon treatment
with PED antisense or control oligonucleotides.

| PED relative abundance | no addition | control oligo | PED-AS oligo |
|---|---|---|---|
| RIN-1 | 100 | 95 ± 8 | 21 ± 4 * |
| U373MG | 100 | 92 ± 9 | 24 ± 5 * |

* Differences among values were analyzed by t-test and were statistically significant (p < 0.01)

1. A method for the diagnosis or prognosis of papilloma or for the determination of the risk to develop a papilloma, comprising the determination of the level and/or the activity of:
(a) a transcription product of a gene coding for PED/PEA-15, and/or
(b) a translation product of a gene coding for PED/PEA-15, and/or
(c) a fragment or derivative of said transcription or translation product,
in a sample coming from a subject, to whom a papilloma is to be diagnosed or prognosticated, comparing said level and/or activity to a reference value representative of a papilloma or health status, then formulating a diagnosis or a prognosis of papilloma in said subject or determining if said subject is at risk of developing a papilloma.

2. A method for monitoring the progression of a papilloma in a subject, comprising the determination of the level and/or activity of:
(a) a transcription product of a gene coding for PED/PEA-15, and/or
(b) a translation product of a gene coding for PED/PEA-15, and/or
(c) a fragment or derivative of said transcription or translation product,
in a sample coming from said subject and comparing said level and/or activity to a reference value representative of a papilloma or health status, then monitoring the progression of papilloma.

3. A method for the evaluation of a therapeutic treatment of a papilloma in a subject, comprising the determination of the level and/or activity of:
(a) a transcription product of a gene coding for PED/PEA-15, and/or
(b) a translation product of a gene coding for PED/PEA-15, and/or
(c) a fragment or derivative of said transcription or translation product,
in a sample coming from said subject and comparing said level and/or activity to a reference value representative of a papilloma or health status, then evaluating the treatment of papilloma.

4. The method according to claim 1, wherein said sample is a skin sample.

5. The method according to claim 1, wherein said reference value is the value of the level and/or activity of:
(a) a transcription product of a gene coding for PED/PEA-15, and/or
(b) a translation product of a gene coding for PED/PEA-15, and/or
(c) a fragment or derivative of said transcription or translation product,
in a sample coming from a subject who is not affected by papilloma.

6. The method according to claim 1, wherein said transcription and/or translation product and/or fragment or derivative is, respectively, mRNA and/or the PED/PEA-15 protein and/or a fragment or a derivative thereof.

7. The method according to claim 1, wherein said transcription product and/or fragment or derivative is determined by means of PCR or Northern blot analysis.

8. The method according to claim 1, wherein said translation product and/or fragment or derivative is determined by means of an immune assay, an enzyme activity assay and/or a binding assay.

9. The method according to claim 1, further comprising the comparison with a level and/or activity of
(a) a transcription product of a gene coding for PED/PEA-15, and/or
(b) a translation product of a gene coding for PED/PEA-15, and/or
(c) a fragment or derivative of said transcription or translation product,
in a series of samples coming from said subject and collected in a period of time.

10. The method according to claim 9, wherein said subject receives a therapeutic treatment before the collection of one of the periods of time.

11. The method according to claim 9, wherein said level and/or activity is determined before and after the treatment of said subject.

12. A kit for the diagnosis or prognosis of papilloma or for the determination of the risk of developing a papilloma or for the monitoring of the progression of a papilloma or for the evaluation of a therapeutic treatment of a papilloma comprising a transcription product of a gene coding for PED/PEA-15, and/or a translation product of a gene coding for PED/PEA-15, and/or a fragment or derivative of said transcription or translation product.

13. A non-human transgenic animal comprising a non-native genetic sequence coding for PED/PEA-15, or a fragment or a derivative thereof, its progeny and different transgenic lines.

14. The animal according to claim 13, expressing PED/PEA-15 ubiquitously.

15. The animal according to claim 13, expressing PED/PEA-15 specifically or preferentially in a particular tissue.

16. The animal according to claim 13, which is mammal.

17. The animal according to claim 16, which is a mouse.

18. The animal according to claim 13, wherein a disruption of said gene results in a predisposition to developing a papilloma.

19. A method for obtaining the animal of claim 13 comprising:
(a) providing a gene targeting construct comprising said gene sequence and a selectable marker sequence, and
(b) introducing said construct in a stem cell of a non-human animal, and
(c) introducing said stem cell in a non-human embryo, and
(d) transplanting said embryo in a non-human pseudopregnant animal, and
(e) allowing said embryo to develop to term, and
(f) identifying a genetically altered non-human animal whose genome comprises a modification of said gene sequence in both alleles, and
(g) breeding said genetically altered animal to obtain a non-human animal whose genome comprises a modification of said endogenous gene.

20. An animal obtainable by the method of claim 19.

21-24. (canceled)

25. An assay for the screening of a substance useful for the treatment of papilloma comprising:
(a) contacting a biological model of papilloma with said substance;
(b) measuring the activity and/or the level of a second substance selected in the group consisting in a transcription product of a gene coding for PED/PEA-15, and/or a translation product of a gene coding for PED/PEA-15, and/or a fragment or derivative of said transcription or translation product,
(c) measuring the activity and/or the level of said second substance in a control biological sample, which was not contacted with said substance;
(d) comparing the activities and/or levels of steps (b) and (c) and determine whether said substance is a inhibitor of said second substance.

26. The assay according to claim 25, wherein said model of papilloma is the animal of claim 13, or any part of it.

27. A substance obtainable by the assay of 25 for the preparation of a medicament for the prevention and/or treatment of papilloma.

28. An antisense oligonucleotide targeted to nucleobase 1 to nucleobase 100 of a nucleic acid molecule encoding PED/PEA-15.

29. The antisense oligonucleotide according to claim 28, said oligonucleotide being targeted to sequences encompassing nucleobase 70, 71 or 72 of a nucleic acid molecule encoding PED/PEA-15.

30. The antisense oligonucleotide according to claim 28, wherein said oligonucleotide is 8 to 30 nucleobases in length.

31. The antisense oligonucleotide according to claim 28 selected from the group consisting of 5′-tgacgcctccggagctgaga-3′ and 5′-tgacgcctctggagctgagc-3′.

32. The antisense oligonucleotide according to claim 28, wherein the modified internucleoside linkage is a phosphorothioate linkage and/or the antisense oligonucleotide comprises at least one modified sugar moiety and/or antisense oligonucleotide comprises at least one modified nucleobase.

33. The antisense oligonucleotide according to claim 32, wherein the modified sugar moiety is a 2′-o-methoxyethyl sugar moiety.

34. The antisense oligonucleotide according to claim 32, wherein the modified nucleobase is a 5-methylcytosine.

35. A method of preparing a medicament comprising formulating a oligonucleotide of claim 28 as a medicament.

36-39. (canceled)

40. A pharmaceutical composition comprising at least a substance of claim 27 in admixture with at least a pharmaceutically acceptable vehicle or excipient.

41. The pharmaceutical composition according to claim 40, suitable for topical administration.

42. The pharmaceutical composition according to claim 40, further comprising at least an antitumor active ingredient.

43. The pharmaceutical composition according to claim 42, wherein said antitumor active ingredient is TRAIL.

44. (canceled)
